(19) United States
(12) Patent Application Publication
(10) Pub. No.: US 2024/0058013 A1
(43) Pub. Date: Feb. 22, 2024

(54) Embolisation System for Promoting Blood Clot Formation

(71) Applicant: Clearstream Technologies Limited, Enniscorthy, County Wexford (IE)

(72) Inventors: Allan Ronan, Enniscorthy (IE); Ciaran Giles, Celbridge (IE)

(21) Appl. No.: 18/260,875

(22) PCT Filed: Jan. 12, 2021
(86) PCT No.: PCT/EP2021/050451

§ 371 Date: Jul. 10, 2023

(51) Int. Cl. A61B 17/12

(52) U.S. Cl. CPC A61B 17/12145; A61B 17/12109; A61B 2017/12054; A61B 2017/00526

(57) ABSTRACT

An embolisation system comprising: an embolisation device (100) for promoting clot formation in a lumen comprising a stem (110) and a plurality of flexible bristles (120) extending outwardly from the stem (110), the bristles (120) having a collapsed delivery configuration and a deployed configuration in which the bristles (120) extend at least radially outwardly from the stem (110) to anchor the embolisation device (100) in a lumen; and a delivery element (150) connected to the stem (110) of the embolisation device (100) via a detachment element (140), wherein the detachment element (140) is configured to break upon application of a predetermined amount of force.

### TECHNICAL FIELD

The present disclosure relates to an embolisation system for promoting clot formation in a bodily lumen, comprising an embolisation device having a collapsed delivery configuration and an expanded deployed configuration for anchoring the embolisation device in the bodily lumen. The present disclosure also relates to a method of manufacturing an embolisation system.

### BACKGROUND

Embolisation devices may be deployed in the vasculature at a particular location by a medical practitioner so as to promote blood clot formation and ultimately occlude the blood vessel. Typically, an embolisation device is pushed through a guide catheter using a delivery wire until a point of deployment is reached. Once the device reaches the required point of deployment, the device is deployed from the guide catheter, detached from the delivery wire, and the delivery wire is removed.

The path of the embolisation device through the guide catheter to the point of deployment is typically tortuous, wherein the delivery wire and the embolisation device may be torqued and twisted along the path. As such, relative rotation and strain between the delivery wire and the embolisation device may be experienced. This can potentially lead to pre-mature detachment of the device from the delivery wire before the point of deployment is reached.

In view of the above, there is a need for an improved embolisation system which is able to deploy the embolisation device at the desired location whilst minimising the probability of premature detachment of the device.

Alternatively or additionally, there is a need for providing an improved embolisation system that is simple to manufacture and/or to deploy, whilst keeping the probability of premature deployment minimised.

### SUMMARY

According the a first aspect of the present disclosure, there is provided an embolisation system comprising: an embolisation device for promoting clot formation in a lumen comprising a stem and a plurality of flexible bristles extending outwardly from the stem, the bristles having a collapsed delivery configuration and a deployed configuration in which the bristles extend at least radially outwardly from the stem to anchor the embolisation device in a lumen; and a delivery element connected to the stem of embolisation device via a detachment element, wherein the detachment element is configured to break upon application of a predetermined amount of force. The force may be a linear separating force (e.g. longitudinal) or a rotation or shearing force. The detachment element may allow the embolisation device to be detachable from the delivery element in a predictable manner whilst simplifying the number of components required for the detach system. The detachment element may allow for a lower delivery profile of the embolisation device in the collapsed delivery configuration.

The delivery element may be a delivery wire.

The detachment element may be a shearable element configured to break upon a predetermined rotation of the delivery element relative to the stem. For example, the shearable element may be configured to break upon at least 6, 8, 10, 12 or more full rotations of the delivery element relative to the stem. The predetermined rotation can be selected such that the expected amount of rotation between the delivery element and the stem during delivery of the embolisation device to a deployment site is less than the predetermined rotation. As such the risk of premature detachment may be reduced.

The detachment element may comprise a necked portion. A cross-sectional width of the necked portion may be 50% or less of the cross-sectional area of the delivery element. This ensures preferential breaking of the detachment element when the force is applied. The detachment element may comprise a weakening structure such as a fracture. A cross-sectional area of the weakening structure may be 50% or less of the cross-sectional area of the delivery element. The detachment element may comprise a first material and the stem a second material, wherein the first material is different from and less stiff than the second material. The first material may be Nylon, PTFE, or Cobalt-chrome and the second material may be nitinol or Cobalt-chrome. The detachment element may comprise any combination of necked portions, weakening structures and less stiff materials. Necked portions, weakening structures and sections having a different material to the material of the stem may be simpler to manufacture and to deploy than other detach mechanisms.

The embolisation device may further comprise a membrane disposed on the stem, configured to restrict flow through the bodily lumen, having a collapsed delivery configuration and a deployed configuration. This may result in quicker blood clot formation and improved embolisation.

The embolisation device may be configured such that in the collapsed delivery configuration, the bristles and/or the membrane overlap with the detachment element. Advantageously, the overlapping bristles and/or membrane may inhibit premature detachment of the embolisation device from the delivery element. The embolisation device may also have a low profile at the overlapping point of the bristles and/or membrane and the detachment element in the collapsed delivery configuration.

According to a second aspect of the disclosure, there is provided a method of manufacturing an embolisation system including an embolisation device for promoting clot formation in a lumen, comprising: providing a stem; providing a delivery element; providing a detachment element between the stem and the delivery element, the detachment element configured to break upon application of a predetermined amount of force; and attaching a plurality of bristles to the stem, extending outwardly from the stem, the bristles having a collapsed delivery configuration and a deployed configuration in which the bristles extend at least radially outwardly from the stem to anchor the embolisation device in a lumen. The detachment element may comprise a necked portion, a weakening structure (optionally a fracture) or a first material, the stem and delivery element comprising second materials each different from the first material, wherein the first material is less stiff than the second materials. Advantageously, the method of manufacture may be simpler than other methods by reducing the number of parts required for a detach mechanism.

### BRIEF DESCRIPTION OF THE DRAWINGS

To enable a better understanding of the present disclosure, and to show how the same may be carried into effect, reference will now be made, by way of example only, to the accompanying schematic drawings, in which:

FIG. 1 schematically depicts a side view of an embolisation device according to the present disclosure in an expanded deployed configuration, according to one or more embodiments shown and described herein;

FIG. 2A schematically depicts a side view of an embolisation device according to the present disclosure in a collapsed delivery configuration, according to one or more embodiments shown and described herein;

FIG. 2B schematically depicts a side view of an embolisation device according to the present disclosure in an expanded deployed configuration before detachment from a delivery wire, according to one or more embodiments shown and described herein;

FIG. 2C schematically depicts a side view of an embolisation device according to the present disclosure in an expanded deployed configuration after detachment from a delivery wire, according to one or more embodiments shown and described herein;

FIG. 3 schematically depicts a side view of another embolisation device according to the present disclosure in a collapsed delivery configuration, according to one or more embodiments shown and described herein;

FIGS. 4A to 4C schematically depict side views of exemplary detachment elements according to according to one or more embodiments shown and described herein; and

FIGS. 5A to 5E schematically depict various methods of manufacturing an embolisation system according to embodiments shown and described herein.

### DETAILED DESCRIPTION

Throughout this disclosure the term ‘embolisation device’ may refer to a device which may be permanently or semi-permanently implanted in a bodily lumen. Accordingly, the ‘embolisation device’ may be configured to be disposed within the bodily lumen for a period of time, such as a number of days, or disposed in the lumen indefinitely. To this end, the ‘embolisation device’ may be configured to be selectively detached from a delivery element so that it may be implanted in the bodily lumen in isolation.

Throughout this disclosure the term ‘bodily lumen’ may refer to the inside space within a tubular structure of the human or animal body. The ‘bodily lumen’ may be, for example, an artery or vein.

Throughout this disclosure the term ‘collapsed delivery configuration’ of an element may refer to a configuration of the element which has a smaller radial extent than an expanded deployed configuration of the element.

Throughout this disclosure the term ‘to anchor’ may refer to partly or fully securing an element in a position.

Throughout this disclosure the term ‘detachment element’ may refer to any element that is configured to break upon application of a predetermined amount of force. More specifically, it may refer to any element which is configured to break irreversibly such that two elements which are connected by the detachment element are separated irreversibly.

Throughout this disclosure, the term ‘shearable element’ may refer to any element that is configured to break irreversibly under a predetermined amount of shear applied.

Throughout this disclosure the term ‘necked portion’ may refer to an elongated portion having a radial extent that is less than the radial extent of another portion.

Throughout this disclosure the term ‘weakening structure’ may refer to any irregularity in an element (for example a geometrical irregularity in the cross-sectional area of the element) configured to reduce the amount of shear required to break the element.

Throughout this disclosure, the term ‘stem’ may refer to an elongate element which extends longitudinally along the length of the embolisation device to act as a backbone for the device, and has a significantly smaller radial extent than the further elements of the embolisation device (for example, the plurality of flexible bristles). The stem may extend along substantially the whole longitudinal extent of the plurality of flexible bristles (e.g. when the embolisation device is in an unrestrained configuration, collapsed delivery configuration and/or expanded deployed configuration). The stem may extend along substantially the whole length of the embolisation device.

In any of the embodiments described herein, the term ‘bristle’ may refer to an elongate strand of material formed substantially a single piece. The ‘bristle’ may be a resilient bristle. The resilient bristle may be biased towards a particular curvature.

Throughout this disclosure, the term ‘radially outwardly’ does not exclude the element additionally extending in the longitudinal direction of the device. For example, the plurality of flexible bristles may extend radially outwardly and longitudinally from the stem.

Through this disclosure, the term ‘profile’ may refer to a radial extent of the embolisation system or device.

Throughout this disclosure, the term ‘bristle segment’ may refer to a group of bristles wherein the spacing between adjacent bristles is less than a predetermined distance. When two bristle segments are “spaced apart”, the spacing between the bristle segments (i.e. the spacing between the most distal bristle of the first segment and the most proximal bristle of the second segment) is greater than the spacing between adjacent bristles within at least one of the bristle segments.

The plurality of flexible bristles may have a collapsed configuration in the collapsed delivery configuration. The plurality of flexible bristles may have an expanded configuration in the expanded deployed configuration. The plurality of bristles may extend radially outwardly from the stem in a plurality of circumferential directions about the stem.

In the expanded configuration, the plurality of flexible bristles may be configured to anchor the device in the bodily lumen. The plurality of flexible bristles may be configured to provide substantially all of the anchoring force for the embolisation device in the bodily lumen.

In the expanded configuration, the plurality of flexible bristles may be configured to contact the bodily lumen.

The device adopts the collapsed delivery configuration when the device is positioned inside the delivery catheter. More particularly, in the collapsed delivery configuration, the plurality of flexible bristles extending outwardly from the core or stem have a radial extent which is less than the radial extent of the bristles in the expanded deployed configuration of the element.

The bristles may be made of a flexible or resiliently deformable material such as stainless steel, Elgiloy or Nitinol. Other suitable materials may also be used, such as any suitable polymer or any other shape memory metal or metal alloy. The flow restrictor may be a thin film membrane made of a self-expanding material such as a polymer, stainless steel or Nitinol. The stem may be made of stainless steel or other suitable material and may comprise a twisted wire from which the bristles extend and on which the flow restrictor is mounted. The stem may alternatively comprise a hollow tube wherein the walls of the hollow tube hold the radially extending bristles in place. For example, the tube may be formed from a heat shrinkable material. Alternatively or additionally, the bristles may be held by the stem using other means such as adhesives.

The diameter of an individual flexible bristle may range from 0.036 mm (0.0014 inches) to 0.053 mm (0.0021 inches). For example, the diameter of an individual flexible bristle may be 0.0381 mm (0.0015 inches), 0.0445 mm (0.00175 inches) or 0.0508 mm (0.002 inches). The average radial diameter of the radial profile formed by the expanded flexible bristles may range from 5 mm to 30 mm.

A flow restrictor may be a membrane made from thin film Nitinol, thin film PTFE, a thin film elastomer such as polyurethane or any other type of suitable biocompatible material. The membrane may have a thickness of 4 μm to 35 μm and a radial diameter of 5 mm to 20 mm. For example, the diameter of the membrane may be 6.5 mm, 9 mm or 16 mm. Furthermore, the membrane may be non-permeable or semi-permeable.

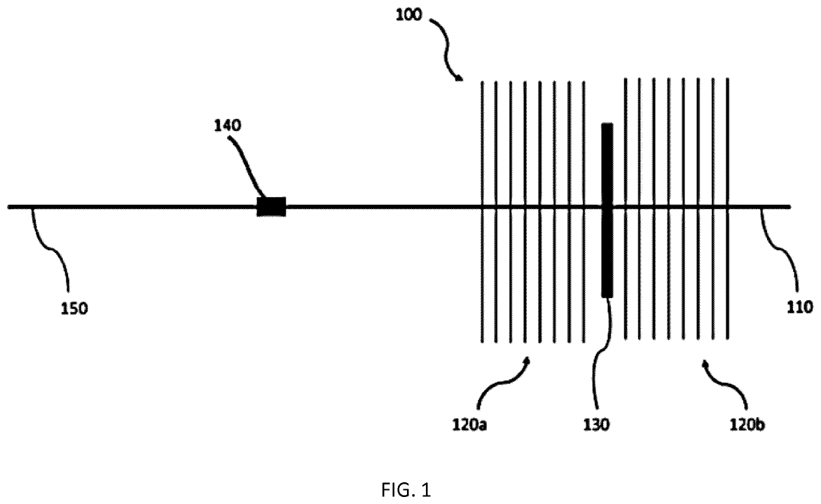

FIG. 1 shows an embolisation system. The embolisation system comprises an embolisation device 100 configured for deployment in a bodily lumen so as to promote clot formation therein, connected to a delivery wire 150 via a detachment element 140 (for example, a shearable element). The embolisation device 100 in FIG. 1 is shown in an unconstrained (i.e. expanded) configuration. It is noted that whilst the detachment element 140 is shown in the figures as having a larger radial profile than the delivery wire 150 and stem 110, this is only for illustrative purposes and the radial profile of the detachment element 140 may in fact have a similar or lower radial profile than the delivery wire 150 and stem 110.

The embolisation device 100 comprises a stem 110, a plurality of flexible bristles 120a extending radially outward around the stem, 120b and a flow restricting membrane 130. In any of the embodiments described herein, the flow restricting membrane 130 is optional. Furthermore, there may be a single group of bristles or a plurality of longitudinally separated groups or segments of bristles. The position of the flow restricting membrane 130 may also vary in different embodiments. For example, in the illustrated embodiment, the membrane 130 is located between two bristle segments 120a and 120b, whereas in other embodiments, the membrane 130 may be positioned proximal to the bristles 120a, distal to the bristles 120b, or located longitudinally within the bristles 120a or 120b.

The stem 110 extends along the longitudinal length of the embolisation device 100. The stem 110 may extend along substantially the whole longitudinal length of the embolisation device 100 when the embolisation device 100 is in the unconstrained configuration.

The stem 110 may be flexible, for example, flexible along substantially its entire length. The stem 110 may be flexible such that the embolisation device 100 when deployed in a bodily lumen conforms to the shape of the bodily lumen. The stem 110 may have flexible sections, hinges and/or connectors (not shown) disposed along its length. Additionally or alternatively, the stem 110 may have a pre-curved shape.

A portion of the stem 110, for example, a proximal portion, is attached to delivery element 150 (such as a delivery wire) via a detachment element 140. Whereas in some embolisation systems the embolisation device is connected to the delivery element via a screw mechanism, the present disclosure relates to the two being connected by a detachment element 140. The detachment element 140 may provide a detachment mechanism that is simpler to manufacture and has a lower profile than a detachment mechanism comprising a screw mechanism or similar structure (a screw mechanism requires a male and female screw thread which increases the profile of the system). Furthermore, a detachment element as described herein breaks after a certain amount of rotation in either a clockwise or counter-clockwise direction, in contrast to a screw mechanism which only detaches in response to rotation in a single direction. As such, the detachment process is simplified.

FIG. 1 shows the plurality of flexible bristles 120a, 120b as two spaced-apart segments of bristles in the form of a proximal bristle segment 120a and a distal bristle segment 120b which are spaced apart along the longitudinal length of the stem 110. However, as would be understood by the skilled person, various arrangements of the plurality of flexible bristles 120a, 120b is possible, for example, in any number of segments, including a single segment. Furthermore, the plurality of flexible bristles 120a, 120b need not be identical and may have, for example, different lengths, materials, flexibilities and/or thicknesses.

Each of the plurality of flexible bristles 120a, 120b is secured to the stem 110 and extends radially outwardly from the stem 110.

Each of the plurality of flexible bristles 120a, 120b may be spaced apart along the longitudinal length of the stem 110. In certain embodiments, at least some of the plurality of flexible bristles 120a, 120b may be disposed at the same axial location along the stem 110.

The flow restricting membrane 130 may be attached to the stem 110. The flow restricting membrane 130 may have a hole therein through which the stem 110 passes, however, other arrangements are contemplated.

The flow restricting membrane 130 may extend radially outwardly from the stem 110. The flow restricting membrane 130 may be of any shape, for example, generally circular. The flow restricting membrane 130 may be flexible, resilient and/or pre-curved. In alternative embodiments, the flow restricting membrane 130 may be any kind of flow restrictor.

The flow restricting membrane 130 may be disposed between some of the plurality of flexible bristles 120a, 120b. In certain embodiments, the flow restricting membrane 130 may be disposed between the proximal bristle segment 120a and the distal bristle segment 120b, as shown in FIG. 1.

The stem 110 may comprise a twisted flexible wire wherein the twisted wire holds the radially extending bristles in place.

The wire may, for example, be made of a flexible twisted metal wire such as stainless steel. In some embodiments, the diameter of the wire used may be 0.5 mm or less, though other diameters are contemplated and possible. The stem 110 may alternatively comprise a hollow tube wherein the walls of the hollow tube hold the radially extending bristles in place. For example, the tube may be formed from a heat shrinkable material. Alternatively or additionally, the bristles may be held by the stem using other means such as adhesives.

The flexible bristles 120a, 120b may be made from Nitinol, Elgiloy or stainless steel or any other shape-memory metal or polymer. The diameter of an individual flexible bristle 32 may range from 0.036 mm (0.0014 inches) to 0.053 mm (0.0021 inches). For example, the diameter of an individual flexible bristle 32 may be 0.381 mm (0.015 inches), 0.445 mm (0.0175 inches) or 0.508 mm (0.02 inches). The overall radial diameter of the expanded flexible bristles 120a, 120b may range from 5 mm to 30 mm.

The membrane 130 may be made from thin film Nitinol, thin film PTFE, a thin film elastomer such as polyurethane or any other type of suitable biocompatible material. The membrane 36 may have a thickness of 4 μm to 35 μm and a radial diameter of 5 mm to 20 mm. For example, the diameter of the membrane 36 may be 6.5 mm, 9 mm or 16 mm. Furthermore, the membrane 36 may be non-permeable or semi-permeable.

The delivery element 150 is securely attached to the detachment element 150 at a proximal end of the detachment element. The stem 110 of the embolisation device 100 is securely attached to the detachment element 150 at a distal end of the detachment element. The delivery element 150 and the stem 110 may be attached to the detachment element by any suitable attaching means, such as by adhesive, welding, interference fit or crimping.

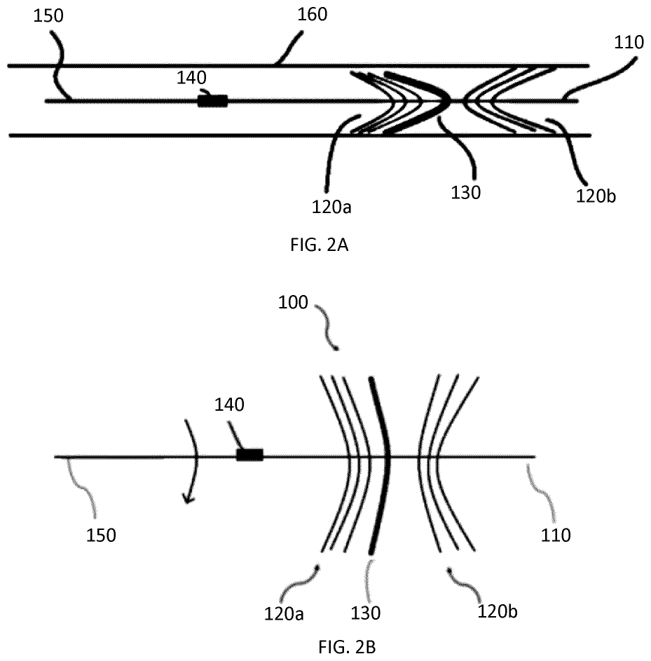

The process of deploying an embolisation device 100 according to the present disclosure is now described with reference to FIGS. 2A to 2C.

FIG. 2A shows a side view of the embolisation system in a collapsed delivery configuration disposed inside a catheter 160. In the collapsed delivery configuration the radial extent of the embolisation device 100 is smaller than in the expanded deployed (unrestricted) configuration, shown in FIG. 1.

The flexible bristles 120a, 120b and the membrane 130 are also in their collapsed delivery configuration, where their radial extent is smaller than in the expanded deployed configuration.

The collapsed delivery configuration is suitable for delivering the embolisation device 100 into a bodily lumen to a deployment location or retrieving the embolisation device 100 from the bodily lumen. The radial extent of the embolisation device 100 is small enough so that it can fit inside the lumen of the catheter 160 to deliver it to a desired site in the bodily lumen.

In the collapsed delivery configuration of FIG. 2A, the first plurality flexible bristles 120a point in a proximal direction and the second plurality of flexible bristles 120b point in a distal direction. The longitudinal orientation of the flexible bristles 120a, 120b does not change when the embolisation device 10 is transitioned from the collapsed delivery configuration to the expanded deployed configuration. This allows the embolisation device 100 to inhibit movement within the bodily lumen in both directions. It will be appreciated that in other embodiments, the flexible bristles of the embolisation device 100 all point in the distal or the proximal direction, or the flexible bristles comprise multiple groups of bristles each pointing in either the proximal or distal directions. Whilst the membrane 130 is shown as being oriented in the proximal direction, in other embodiments the membrane points in the distal direction. In embodiments where the membrane 130 is located longitudinally within a group of bristles, the membrane 130 points in the same longitudinal direction as the adjacent bristles in the contracted delivery configuration.

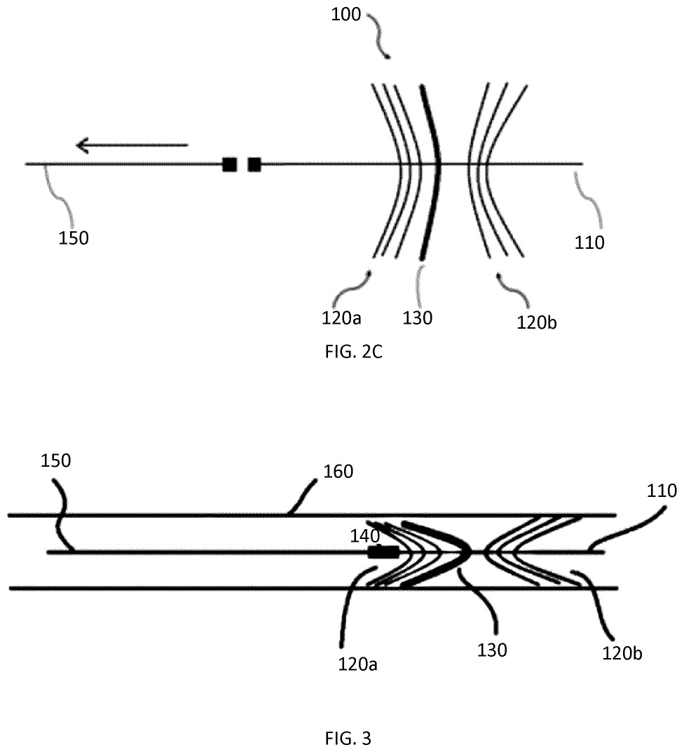

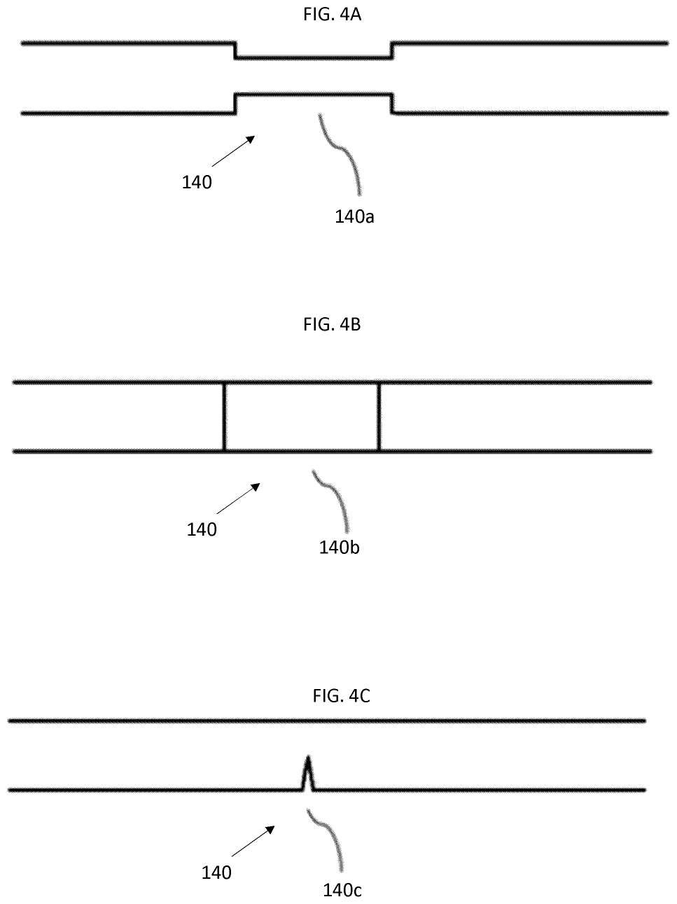

FIG. 2A shows an embodiment wherein the bristles 120a, 120b and/or membrane 130 do not overlap longitudinally with the detachment element 140 in the collapsed delivery configuration. In other embodiments, the detachment element 140 is located such that in the collapsed delivery configuration, a group of bristles 120a, 120b and/or the membrane 130 overlap the detachment element (a schematic of one such embodiment in the collapsed delivery configuration is shown in FIG. 3). Such an arrangement may provide additional protection to the detachment element 140 during delivery of the embolisation device 100 through the catheter 160 to a delivery site, inhibiting premature detachment of the embolisation device 100 from the delivery wire 150. Furthermore, the profile of the embolisation device at the point of overlap of the detachment element and the bristles and/or membrane remains low as compared to the overlap profile for other detachment mechanisms, such as a screw mechanism. For example, the detachment element 140 may take the form shown in any of FIGS. 4A to 4C, in which the radial profile of the detachment element is similar to or lower than the stem/delivery element. In contrast, a screw mechanism typical requires components which have a larger radial profile than the delivery element and/or stem of the embolisation device.

In normal operation, the embolisation device 100 is pushed through the delivery catheter 160 in a collapsed delivery configuration such as shown in FIG. 2A until it reaches the intended delivery site in the bodily lumen. Once the distal tip of the embolisation device is at the desired location (i.e. once the distal tip of the embolisation device 100 is at the tip of the catheter 160, which has been located at the site of delivery), then the delivery wire 150 and embolisation device 100 are held still and the delivery catheter 160 is retracted to deploy the embolisation device 100 within the bodily lumen. Once deployed, a combination of the bristles 120a, bristles 120b and membrane 130 move to the expanded deployed configuration in which the embolisation device 100 engages with and anchors to the wall of the bodily lumen.

FIG. 2B shows the embolisation device 100 in the expanded deployed configuration after the catheter 160 has been retracted. In this configuration, the bristles 120a, 120b and membrane 130 engage the wall of a bodily lumen (not shown) to anchor the embolisation device 100 in place. Once it is determined that the embolisation device 100 is in the correct position, the delivery wire 150 is rotated by a predetermined amount, in either direction, about the longitudinal axis of the delivery wire 150. The rotation can be effected by a user at the proximal end of the delivery wire, using any suitable mechanism. As the embolisation device 100 is anchored to the bodily lumen, relative rotation between the delivery wire 150 and the embolisation device 100 occurs. This rotation results in an increased amount of torque being applied at the detachment element 140. Once sufficient torque is applied to the detachment element 140, the detachment element breaks. In any of the embodiments disclosed herein, the force required to break the detachment element 140 can be selected to be low enough such that the embolisation device 100 is not dislodged or moved from the anchored position in the bodily lumen when applying the force to break the detachment element 140. This breaking force can be selected based on the anchoring properties of the particular embolisation device 100 being used, which is determined by, for example, the number and type of bristles of the embolisation device 100.

FIG. 2C shows the embolisation device 100 after the detachment element 140 breaks from rotation of the delivery wire 150. The embolisation device 100 is no longer connected to either the catheter 160 or the delivery wire 150 and is held in place within the bodily lumen (not shown) by the anchoring force of the bristles 120a, 120b and/or the membrane 130. The embolisation device 100 is therefore fully deployed and the delivery wire 150 is retracted from the bodily lumen.

Different possible types of detachment or elements will now be described with reference to FIGS. 4A to 4C.

FIG. 4A shows a detachment element 140 according to one embodiment of the present disclosure. The detachment element 140 of FIG. 4A comprises a necked portion 140a connecting the delivery wire 150 and stem 110 of the embolisation device 100. The necked portion has a radial extent that is less than both the radial extent of the delivery wire 150 and the stem 110 of the embolisation device 100. The necked portion is prone to a high amount of twist when torque is applied to the delivery wire 150. As a result, after a sufficient amount of torque is applied to the delivery wire 150, the necked portion 140a breaks and the stem 110 is separated from the delivery wire 150. The amount of torque required to break the necked portion 140a (i.e. the amount of rotation of the delivery wire 150) is determined by the dimensions of the necked portion 140a and the material of the necked portion 140a. The dimensions can be selected such that the necked portion 140a is configured to shear upon an amount of rotation of the delivery wire 150 that is above the expected amount of relative rotation of the device during the delivery process (due to the tortuous path taken by the device through the delivery catheter). For example, the detachment element may be made of Nylon, PTFE, or Cobalt-chrome and a cross-sectional area of the necked portion may be selected to be 50% or less of the cross-sectional area of the delivery element.

FIG. 4B shows a detachment element 140 according to another embodiment of the disclosure. The detachment element 140 of FIG. 4B comprises a portion 140b that is a different material from the material of the delivery wire 150 and the material of the stem 110. In particular, the material of portion 140b is selected to be a material which is less stiff than the material of the delivery wire 150 and the stem 110. As such, any torque applied to the delivery 150 results in a larger amount of twist at the portion 140b. After a sufficient amount of torque is applied to the delivery wire 150, the portion 140b breaks and the stem 110 is separated from the delivery wire 150. The amount of torque required to break the portion 140b (i.e. the amount of rotation of the delivery wire 150) is directly determined by the material properties of the portion 140b, the delivery wire 150 and the stem 110. The materials can be selected such that the portion 140b is configured to shear upon an amount of rotation of the delivery wire 150 that is above the expected amount of relative rotation of the device during the delivery process (due to the tortuous path taken by the device through the delivery catheter). For example, the material of the portion 140b may be selected from Nylon, PTFE, or Cobalt-chrome, and the materials of the delivery wire 150 and the stem 110 may be selected from nitinol or Cobalt-chrome (such that the material of the portion 140b differs).

FIG. 4C shows a detachment element 140 according to another embodiment of the present disclosure. The detachment element 140 of FIG. 4C comprises a weakening structure 140c connecting the delivery wire 150 and stem 110 of the embolisation device 100. The weakening structure 140c comprises an irregularity such that the shearing force required to break it is lower than the shearing force required to break the delivery wire 150 and stem 110. As illustrated in FIG. 4C, the weakening structure 140c may be a fracture. The fracture has a radial extent that is less than both the radial extent of the delivery wire 150 and the stem 110 of the embolisation device 100. The weakening structure 140c is prone to a high amount of twist when torque is applied to the delivery wire 150. As a result, after a sufficient amount of torque is applied to the delivery wire 150, the weakening structure 140c breaks and the stem 110 is separated from the delivery wire 150. The amount of torque required to break the weakening structure 140c (i.e. the amount of rotation of the delivery wire 150) may directly determined by the dimensions of the fracture, the delivery wire 150 and the stem 110. The relative dimensions can be selected such that the weakening structure 140c is configured to shear upon an amount of rotation of the delivery wire 150 that is above the expected amount of relative rotation of the device during the delivery process (due to the tortuous path taken by the device through the delivery catheter). The detachment element may be made of Nylon, PTFE, or Cobalt-chrome and may be the same or different material to the delivery wire 150 and/or stem 100. A cross-sectional area of the detachment element 140 may be selected to be 50% or less of the cross-sectional area of the delivery element.

In yet further embodiments, combinations of the features shown in FIGS. 4A to 4C are shown. For example, a necked portion or weakening structure having a different material to the material of the delivery wire 150 and stem 110 may be used, and so on. The amount of torque required to break the shearable element would be readily determinable by the skilled person by testing.

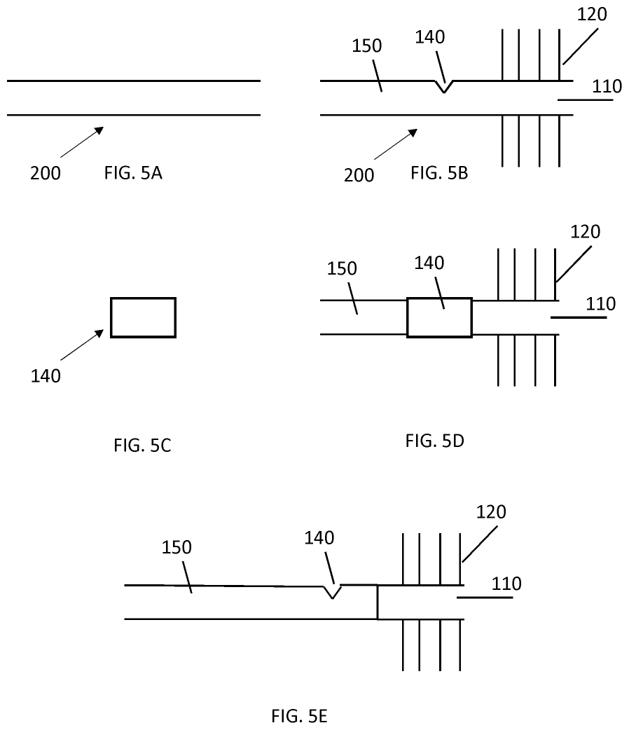

As depicted in FIGS. 5A and 5B, the detachment element 140 may be manufactured by machining or molding a necked portion or fracture into an elongate element 200. A first side of the elongate element 200 relative to the detachment element 140 may be considered the delivery element 150 and the opposite side of the elongate element 200 may be the stem 110 on which the bristles 120 are mounted (the bristles and any other elements of the embolisation device may be mounted before or after manufacture of the detachment element, by welding, adhesive or otherwise).

Alternatively, as shown in FIGS. 5B and 5C, the detachment element may be manufactured as a separate element, with respective ends of the elongate element 140 attached to a proximal end of the stem 110 of an embolisation device and a distal end of a delivery element 150, for example by adhesive, welding or a heat-shrinkable tube.

In yet a further embodiment, as shown in FIG. 5E, the detachment element 140 may be manufactured by machining or molding a distal portion of the delivery element 140 and the delivery element 150 and stem 110 of an embolisation device may be attached, for example by adhesive, welding or a heat-shrinkable tube. Alternatively, the detachment element may be made in a proximal portion of the stem of the embolisation device.

The embolisation system described above may be manufactured by:
providing the stem 110;
providing a delivery element 150;
providing a detachment element 140 between the stem and the delivery element 150; and
attaching a plurality of bristles to the stem, extending outwardly from the stem, the bristles having a collapsed delivery configuration and a deployed configuration in which the bristles extend at least radially outwardly from the stem to anchor the embolisation device in a lumen.

It is noted that the detachment element 140 may be formed in any manner as described above with respect to FIGS. 5A to 5E.

It is noted that the order of the steps given above does not necessarily imply a chronological order. For example, the shearable element 140 could be provided and connected to the stem 110 and/or delivery element 150 before or after the plurality of bristles are attached to the stem 110, or after a first group of bristles are attached to the stem 100 but before a second group of bristles are attached to the stem 100.

The bristles may be attached to the stem by providing two arms of parallel flexible wire (for example two separate wires or one continuous wire bent to form two parallel arms), placing the bristles between the arms of wire, fixing the arms of wire at one end and twisting the arms of wire such that the bristles are held between twists in the wire. The arms of wire may then be fixed together at terminal ends. The shearable element 140 could be attached to flexible wire before or after the bristles are attached by this twisting method.

Alternatively, the stem may comprise a tube having a tube wall, and the bristles may be placed to penetrate the tube wall such that they extend radially outwardly from the tube.

Table 1 below contains exemplary dimensions for the implant, though other dimensions are contemplated and possible.

TABLE 1

| Membrane thickness | Membrane Diameter | | Bristle Diameter |
|---|---|---|---|
| 4-35 microns | 6.5 | mm | 0.381 mm (0.015″) |
| 4-35 microns | 9 | mm | 0.445 mm (0.0175″) |
| 4-35 microns | 16 | mm | 0.508 mm (0.02″) |

All of the above are fully within the scope of the present disclosure, and are considered to form the basis for alternative embodiments in which one or more combinations of the above described features are applied, without limitation to the specific combination disclosed above.

It will be appreciated by the skilled person that any suitable detachment element that is configured to break upon application of a predetermined amount of force may be used.

In light of this, there will be many alternatives which implement the teaching of the present disclosure. It is expected that one skilled in the art will be able to modify and adapt the above disclosure to suit its own circumstances and requirements within the scope of the present disclosure, while retaining some or all technical effects of the same, either disclosed or derivable from the above, in light of his common general knowledge in this art. All such equivalents, modifications or adaptations fall within the scope of the present disclosure.

1. An embolisation system comprising:
an embolisation device for promoting clot formation in a lumen comprising a stem and a plurality of flexible bristles extending outwardly from the stem, the bristles having a collapsed delivery configuration and a deployed configuration in which the bristles extend at least radially outwardly from the stem to anchor the embolisation device in a lumen; and
a delivery element connected to the stem of the embolisation device via a detachment element, wherein the detachment element is configured to break upon application of a predetermined amount of force;
wherein the detachment element comprises a first material, the stem and delivery element comprise second materials each different from the first material, and wherein the first material is less stiff than the second materials.

2. The embolisation system according to claim 1, wherein the detachment element is a shearable element configured to break upon a predetermined shear of the delivery element relative to the stem.

3. The embolisation system according to claim 2, wherein the shearable element is configured to break upon a predetermined rotation of the delivery element relative to the stem.

4. The embolisation system according to claim 1, wherein the detachment element comprises a necked portion.

5. The embolisation system according to claim 4, wherein a cross-sectional area of the necked portion is 50% or less of the cross-sectional area of the delivery element.

6. The embolisation system according to claim 1, wherein the detachment element comprises a weakening structure, and, optionally, wherein the weakening structure is a fracture.

7. The embolisation system according to claim 6, wherein a cross-sectional area of the weakening structure is 50% or less of the cross-sectional area of the delivery element.

8. (canceled)

9. The embolisation system according to claim 1, wherein the first material is one of: Nylon, PTFE, or Cobalt-chrome.

10. The embolisation system according to claim 1, wherein each of the second materials are one or more of: nitinol or Cobalt-chrome.

11. The embolisation system according to claim 1, wherein in the collapsed delivery configuration, the bristles overlap with the detachment element.

12. The embolisation system according to claim 1, further comprising a flow restrictor, the flow restrictor having a collapsed delivery configuration and a deployed configuration, wherein in the collapsed delivery configuration, the membrane overlaps with the detachment element.

13. The embolisation system according to claim 12, wherein the flow restrictor is a membrane disposed on the stem.

14. A method of manufacturing an embolisation system including an embolisation device for promoting clot formation in a lumen, comprising:
providing a stem;
providing a delivery element;
providing a detachment element between the stem and the delivery element, the detachment element configured to break upon application of a predetermined amount of force; and
attaching a plurality of bristles to the stem, extending outwardly from the stem, the bristles having a collapsed delivery configuration and a deployed configuration in which the bristles extend at least radially outwardly from the stem to anchor the embolisation device in a lumen;
wherein the detachment element comprises a first material, the stem and delivery element comprise second materials each different from the first material, and wherein the first material is less stiff than the second materials.

15. The method according to claim 14, wherein the detachment element comprises one or more of:
a necked portion; and
a weakening structure, optionally wherein the weakening structure is a fracture.
